(19) United States
(12) Patent Application Publication
(10) Pub. No.: US 2015/0359795 A1
(43) Pub. Date: Dec. 17, 2015

(54) HIGH DRUG LOAD PHARMACEUTICAL COMPOSITIONS WITH CONTROLLABLE RELEASE RATE AND PRODUCTION METHODS THEREOF

(71) Applicant: Pharmosa Limited, Taichung City (TW)

(72) Inventors: Yu-De Su, Changhua County (TW); Wen-Chun Lin, Taichung City (TW); Yao-Kun Huang, Taoyuan City (TW)

(21) Appl. No.: 14/738,951

(22) Filed: Jun. 15, 2015

(30) Foreign Application Priority Data
Jun. 16, 2014 (TW) 103120713

(51) Int. Cl. A61K 31/517; A61K 31/245; A61K 9/48; A61K 31/194; A61K 9/20; A61K 9/28; A61K 31/506; A61K 31/198

(52) U.S. Cl. CPC A61K 31/517; A61K 31/506; A61K 31/245; A61K 31/198; A61K 31/194; A61K 9/2095; A61K 9/2893; A61K 9/2866; A61K 9/282; A61K 9/2077; A61K 9/4808

(57) ABSTRACT

High drug load pharmaceutical compositions and production methods thereof are provided. The pharmaceutical composition includes at least one viscous active pharmaceutical ingredient or a pharmaceutically acceptable salts thereof. The at least one viscous active pharmaceutical ingredient accounts for at least 60% by weight of the total solid weight of the pharmaceutical composition. The saturated solution of the at least one viscous active pharmaceutical ingredient has a viscosity of at least 20 centipoises at 25° C. Alternatively, the aqueous solution of the at least one viscous active pharmaceutical ingredient with a concentration lower than 55 wt % has at least a viscosity of at least 10 centipoises at 25° C.

### CROSS-REFERENCE TO RELATED APPLICATION

This application claims the priority benefits of Taiwan application serial no. 103120713, filed on Jun. 16, 2014. The entirety of the above-mentioned patent application is hereby incorporated by reference herein and made a part of this specification.

### BACKGROUND OF THE INVENTION

1. Field of the Invention

The present invention relates to a composition and manufacturing method thereof. More particularly, the present invention relates to a high drug load pharmaceutical composition and manufacturing method thereof. More particularly, the present invention relates to a pharmaceutical composition with controllable release rate and manufacturing method thereof.

2. Description of the Prior Art

Traditional pharmaceutical formulation designs relate to the studies in the characteristics of the drug, in combination with the design of using pre-formulation, and based on the results of absorption, distribution, metabolism and excretion of the drug in the human body, the dosage form(s) to be administrated to the patients will be determined. The solid dosage forms usually need to mix with a variety of excipients to achieve the purposes of rapidly dissolving, very rapidly dissolving, slow dissolving and controlled release in vitro. Different formulation dosage forms will lead to the differences in absorption, distribution, metabolism and excretion within the human body. Different excipients may be needed according to the different pharmaceutical formulation designs, and the pharmaceutically excipients comprise fillers, binders, disintegrants, lubricants, surfactants, glidants, stabilizers, release agents, pH adjustment agents, etc.

Almost all of the instant release tablets must use more than a certain percentage of excipients to control the rapid release rate. Furthermore, the active pharmaceutical ingredients with viscous characteristics are prone to self-sticking, which leads to raise the release time of the drug and lower the drug release rate. Thus, a lot of excipients must be used to achieve the purpose of rapid release.

The prior art of Chinese Patent Application No. 200610073074.3 (Publication No. CN 101053562A) relates to an improvement in the release rate of Otilonium bromide as a drug used for relieving abdominal pain caused by Irritable Bowel Syndrome. The invention uses a lot of excipients, such as 43.33% lactose, 20.67% microcrystalline cellulose, 8% crospovidone, 1.3% magnesium stearate, to increase release rate thereof, and more than 90% of the drug may be dissolved in 5 minutes in vitro so as to relieve spasticity rapidly.

Besides, Taiwan Patent No. 1350184 relates to a tablet formulation containing the active pharmaceutical ingredients ranging from 30% to 80%. For one aspect, the tablet can offer rapid release effects. For another aspect, each tablet unit contains a relatively high amount of active pharmaceutical ingredient Imatinib so as to provide the adult daily dose up to 400-800 mg. Taiwan Patent No. 491845 discloses the (3 crystalline form of Imatinib, which has physicochemical properties of tighter crystalline, good flowability and not hygroscopic. Relatively, the a crystalline form of Imatinib has the physicochemical properties of acicular crystalline, low flowability and hygroscopic. Thus, it is better to manufacture the medicine using the β crystalline form of the compound and adding excipients such as binders, disintegrants, glidants and lubricants to produce the required tablets, so that the active drug of Imatinib may be released immediately. According to the patent of high dosage load tablet, for producing relatively high dosage but small sized tablets, it is difficult to manufacture such tablet with Imatinib because of its characteristics of high brittleness and low milling resistance when made into tablets. Therefore, the pharmaceutical compositions must be made under certain conditions to realize the stable manufacturing procedures, and the complicated requirements including complex formulations, complicated processes and spraying film coating on tablets at low temperatures. For example, the high dosage load pharmaceutical composition may need a variety of excipients such as binders, disintegrants, glidants and lubricants and complicated processes are needed for producing the internal and external phases of the formulations. Finally, a stable pharmaceutical composition may be obtained by applying film coating at low temperatures.

Second, almost all of the controlled release tablets not only need to use excipients above a certain percentage but also use one or more polymer excipients to control the release rate. Using polymer excipients for the released control tablets makes either the formulation design or the process control more difficult. Taking an erosion release formulation, such as Etodolac (U.S. Pat. No. 4,966,768), a water-insoluble analgesic drug for treating arthritis, as an example, because a controlled release coated tablet with a weight of about 730 mg must contain 400 mg active pharmaceutical ingredients, a lot of additional polymer excipients for controlled release must be used, including 18-30% hydroxypropyl methylcellulose and 15-28% ethyl cellulose along with dibasic disodium phosphate as the controlled release modifying agent. Additionally, lactose as the filler, magnesium stearate as the lubricant and talcum are needed. As for Alfuzosin HCl (U.S. Pat. No. 6,149,940), which is a water-soluble drug for treating benign prostatic hyperplasia, except for the two layers not containing active pharmaceutical ingredients, the layer containing the active pharmaceutical ingredients also comprises mannitol and microcrystalline cellulose as fillers, colloidal silica as the glidant and polyvinylpyrrolidone as the binder, other than hydroxypropyl methylcellulose the as the controlled release excipient. Taking a diffusion control release formulation, such as Isosorbide mononitrate (U.S. Pat. No. 4,800,084), a water-soluble drug for treating angina pectoris, as an example, because an inert sugar core taking about 30 wt. % relative to the total weight of the formulation, is used as the core base and then the active pharmaceutical ingredients taking about 50 wt. % of the total weight, along with other excipients are coated by complicated methods on the inert sugar core, a lot of additional polymer excipients for controlled release must be used, including 2.5% hydroxypropyl methylcellulose and 3% ethyl cellulose. Additionally, lactose as the filler, polyethylene glycol and talcum are also needed. As for Fluoxetine HCl (U.S. Pat. No. 5,910,319), which is an enteric-coated controlled release pill with a sugar core as an antidepressant, besides for using a variety of polymer controlled release agents for the coating, the formulation of such enteric-release pill has to include the lubricant, binders and fillers to achieve the effect of controlled release. Taking a controlled release coated tablet produced by squeezing out round granules as an example, as described in U.S. Pat. No. 6,274,171, the controlled release formulation of the antidepressant Venlafaxine HCl also has to include the lubricants, binders and fillers to achieve the effect of controlled release, as well as using a variety of polymer controlled release agents for the final coating.

Furthermore, for the formulation of the compound preparation, it requires formulations and processes for a variety of excipients to achieve the purpose of stability and rapid dissolution. As described in U.S. Pat. No. 6,162,802, for Lotrel (Amlodipine and Benazepril HCl), which is a compound preparation drug for treating hypertension in a relatively low dosage, the two active pharmaceutical ingredients of the compound preparation must be completely separated. The formulation contains up to 12 excipients. Also, one of the two active pharmaceutical ingredients must be made into tablets and film-coated by spraying, and then filled into a capsule with the other active pharmaceutical ingredient that has been granulated. As described in Taiwan Patent No. 1357,823, the improved formulation of Amtrel (Amlodipine and Benazepril HCl) prevents the interaction between the two active pharmaceutical ingredients by using the low moisture manufacturing method, and six excipients, including sodium lauryl sulfate as the surfactant and croscarmellose sodium as the rapid disintegrant, are still needed for promoting the rapid release and absorption. Bigger problems may occur for the formulation of the compound preparation with higher dosage loads, such as drugs used for combination therapy (namely cocktail therapy) for treating AIDS. For example, for the individual dosage for the drugs: Abacavir 300 mg, Lamivudine 300 mg and Zidovudine 300 mg, the drug Stribild® has four active pharmaceutical ingredients, including Elvitegravir 150 mg, Cobicistat 150 mg, Tenofovir disoproxil fumarate 300 mg and Emtricitabine 200 mg. However, the drug Trivizir® has three active pharmaceutical ingredients of 300 mg, 150 mg and 300 mg individually, and two pills of such drug is administered daily, along with foreseeable drug resistance of AIDS. The dosage of the drug Stribild® may have to be increased from one tablet per day to two or more tablets per day. The total weight of all the active pharmaceutical ingredients of the drug Trivizir® is up to 750 mg, and will be increased up to 900 mg if calculated in the highest dosage of each single remedy; while the total weight of all the active pharmaceutical ingredients of the drug Stribild® is also up to 800 mg, and each oral tablet will be weighted over 1,200 mg if excipient probably used is considered. Regarding Trivizir® of U.S. Pat. No. 6,417,191, the tablet formulation contains an active pharmaceutical ingredient of 250 mg along with 250 mg of excipient additionally. Comparing to the similar formulation of Trivizir®, the weight of a tablet will be up to 1,500 mg and the weight of a controlled release dosage form is 1,050 mg. Besides, regarding Stribild® of U.S. Pat. No. 8,592,397, the tablet formulation contains an active pharmaceutical ingredient 250 mg along with additional 250 mg of excipients. Comparing to the similar formulation of Stribild®, the weight of a tablet will be high and up to 1,600 mg. In view of the inconvenience for patients caused by swallowing the large sized solid drugs with high drug loads, especially for those with dysphagia, it is important to develop solid dosage form of high drug loads in smaller sizes.

Accordingly, manufacturing the rapid release dosage form often requires a lot of disintegrants, and also needs fillers, binders and lubricants added therein. But for the controlled release dosage form, a large amount of controlled release polymers is needed for both dissolution release and diffusion controlled release; or a sugar core, which occupies most of the space and weight, is adopted and applied with a complex process of spraying of active pharmaceutical ingredients, finally, one or more layer of the controlled release film coating may be performed. Such formulations and manufacturing processes need to use a large amount of and a variety of excipients, which not only lead to increasing the weight of the dosage form and difficulties in swallowing, but also limit or lower the ratios of the active pharmaceutical ingredients in the composition, which is unable to be increased. And it also becomes an obstacle for viscous drugs because high dosage is often needed for administration.

Furthermore, the compatibility between the active pharmaceutical ingredients and excipients is also a very difficult issue. For example, reducing saccharide is not suitable for the active pharmaceutical ingredient of the drug as prone to the oxidation-reduction reaction; while the excipient, which is harmful to the condition of patients, such as using lactose as fillers will cause potential threats to patients with lactose intolerance. According to European Patent No. 2,374,450 A1, the drug using Flupentixol as the active pharmaceutical ingredient has a higher solubility in the acidic environment, therefore, adding acidic excipients will make the drug unstable. Thus, using alkaline excipients can improve the stability of Flupentixol. The disclosure of all references and patent documents recited herein are incorporated by reference in its entirety.

### SUMMARY OF THE INVENTION

In view of the disadvantages of complex formulations and complicated processes in the prior art, the present invention is directed to simple formulations and simple processes, by using an active pharmaceutical ingredient of which the compound itself possesses a specific viscosity. For example, the viscosity of the saturated aqueous solution of the compound must reach 20 centipoises (cPs) at 25° C. or the viscosity of the aqueous solution of less than 55 wt. % of the compound is at least 10 cPs at 25° C., so that the active pharmaceutical ingredient can be a binder directly and be made into solid granular form. The granules may be further made into microtablets of super high drug load individually or add a trace of pharmaceutically acceptable excipient(s). The microtablets may be selected according to the demand in clinical use. The size of microtablets may be adjusted to control the release rate by way of reducing the volume to increase the total surface area. Additionally, by adjusting the ratio and the formulation of the coating film, the microtablets may be coated so as to control the release rate and effects thereof. The above-mentioned solid dosage forms may be further filled into capsules. The drug compositions of super high dosage load proposed by the present invention may significantly reduce the content of excipients and/or binders contained in the solid dosage forms, and the volume of the solid dosage forms may also be reduced so as to produce super high drug load pharmaceutical compositions. It is more important that the solid dosage forms may be made in different ways to control the release rates depending on different demands, so that optimal absorption rate and scheme of the human body can be achieved.

For achieving the above purposes, the present invention provides a pharmaceutical composition, comprising at least one viscous active pharmaceutical ingredient or a pharmaceutically acceptable salt thereof, the content of the at least one viscous active pharmaceutical ingredient or a pharmaceutically acceptable salt thereof is at least 60 wt. %, relative to the total solid weight of the pharmaceutical composition.

Preferably, the content of the at least one viscous active pharmaceutical ingredient or a pharmaceutically acceptable salt thereof is at least 70 wt. %, relative to the total solid weight of the pharmaceutical composition.

Also preferably, the content of the at least one viscous active pharmaceutical ingredient or a pharmaceutically acceptable salt thereof is at least 80 wt. %, relative to the total solid weight of the pharmaceutical composition.

Preferably, the content of the at least one viscous active pharmaceutical ingredient or a pharmaceutically acceptable salt thereof is at least 90 wt. %, relative to the total solid weight of the pharmaceutical composition.

According to the present invention, said “viscous active pharmaceutical ingredient or a pharmaceutically acceptable salt thereof” is defined as: the viscosity of the saturated aqueous solution of the active pharmaceutical ingredient or the salt thereof must reach 20 cPs at 25° C. or the viscosity of the aqueous solution of the active pharmaceutical ingredient or the pharmaceutically acceptable salt thereof less than 55 wt. % is at least 10 cPs at 25° C., so that the active pharmaceutical ingredient or the salt thereof can be a binder directly and be made into solid granules without binders. The viscosities of saturated aqueous solutions or aqueous solutions with different weight percentage are measured with Rotational viscometers (Brooksfield, USA) at constant temperature 25° C., and the rotation speed is 5-100 rpm.

The at least one viscous active pharmaceutical ingredient or a pharmaceutically acceptable salt thereof may be used to treat or alleviate different kinds of diseases or conditions for human or animals.

Preferably, the at least one viscous active pharmaceutical ingredient or a pharmaceutically acceptable salt thereof may be used to treat or alleviate malignant or nonmalignant hyperplasia or act as a tyrosine kinase inhibitor. Preferably, the active pharmaceutical ingredient is Imatinib or a pharmaceutically acceptable salt thereof. Preferably, the active pharmaceutical ingredient is Imatinib mesylate. More preferably, the active pharmaceutical ingredient may be in the a crystalline form or β crystalline form of Imatinib mesylate.

Preferably, the at least one viscous active pharmaceutical ingredient or a pharmaceutically acceptable salt thereof may be used to treat benign prostatic hyperplasia (BPH) and dysuria or act as a selective alpha-1 adrenoceptor antagonist. Preferably, the active pharmaceutical ingredient is Alfuzosin or a pharmaceutically acceptable salt thereof. More preferably, the active pharmaceutical ingredient is Alfuzosin HCl.

Preferably, the at least one viscous active pharmaceutical ingredient or a pharmaceutically acceptable salt thereof may act as antimuscarinics to treat functional gastrointestinal disorders. Preferably, the active pharmaceutical ingredient is Otilonium or a pharmaceutically acceptable salt thereof. More preferably, the active pharmaceutical ingredient is Otilonium bromide.

Preferably, the at least one viscous active pharmaceutical ingredient or a pharmaceutically acceptable salt thereof may be used to treat liver diseases or act as a nutritional supplement. Preferably, the active pharmaceutical ingredient is amino acid derivative or a pharmaceutically acceptable salt thereof. More preferably, the active pharmaceutical ingredient is L-arginine α-ketoglutarate.

The present invention provides a pharmaceutical compositions, comprising at least one viscous active pharmaceutical ingredient or a pharmaceutically acceptable salt thereof, the content of the at least one viscous active pharmaceutical ingredient is at least 60 wt. %, relative to the total solid weight of the pharmaceutical composition. The pharmaceutical composition is obtained by the following manufacturing method, and the manufacturing method comprising steps of: (i) adding a starting material of the at least one viscous active pharmaceutical ingredient to a granulator, and performing granulation to the starting material to obtain granules of the at least one viscous active pharmaceutical ingredient; and (ii) making the granules of the at least one viscous active pharmaceutical ingredient obtained from the step (i) into a solid dosage form of the pharmaceutical composition individually or mixing the granules of the at least one viscous active pharmaceutical ingredient obtained from the step (i) with at least one pharmaceutically acceptable excipient into the solid dosage form of the pharmaceutical composition. The pharmaceutically acceptable excipient, which may attribute to control the drug release rate, is selected from the group consisting of disintegrants, glidants, lubricants and combinations thereof.

According to the present invention, the content of the pharmaceutically acceptable excipient ranges from 0.1 wt. % to 15 wt. %, relative to the total solid weight of the pharmaceutical composition.

Preferably, the content of the pharmaceutically acceptable excipient ranges from 0.1 wt. % to 10 wt. %, relative to the total solid weight of the pharmaceutical composition.

More preferably, the content of the pharmaceutically acceptable excipient ranges from 0.1 wt. % to 5.0 wt. %, relative to the total solid weight of the pharmaceutical composition.

According to the present invention, the “disintegrant” may be used in the said pharmaceutical composition only if no adverse impacts on the active pharmaceutical ingredient, and the disintegrant may comprise, but not limited to, crospovidone, microcrystalline cellulose, croscarmellose sodium, croscarmellose calcium, crosslinked polyvinylpyrrolidone, sodium carboxyl methylstarch, sodium alginate, starch, low-substituted hydroxypropyl cellulose or combinations thereof.

According to the present invention, the “glidant” may be used in the said pharmaceutical composition only if no adverse impacts on the active pharmaceutical ingredient, and the glidant may comprise, but not limited to, starch, pregelatinized starch, powdered cellulose, silica, colloidal silica, talcum or combinations thereof.

According to the present invention, the “lubricant” may be used in the said pharmaceutical composition only if no adverse impacts on the active pharmaceutical ingredient, and the lubricant may comprise, but not limited to, calcium stearate, polyethylene glycol, sodium lauryl sulfate, stearic acid, talcum, magnesium stearate, hydrogenated vegetable oil or combinations thereof. In the preferable embodiment(s) of the present invention, the lubricant is magnesium stearate.

According to the present invention, the “granulation” means performing granulation to the starting material in the step (i), which comprises applying wet granulation by using a high shear mixer, applying dry granulation by using a roller compactor or applying spray granulation by using a fluidized bed granulator.

Preferably, performing granulation to the starting material in the step (i) includes applying spray granulation to granulate by using a fluidized bed granulator.

The “solid dosage form of the pharmaceutical composition” mentioned in the present invention may exist in all kinds of dosage forms of the pharmaceutical composition depending on the clinical demands. The forms comprise, but not limit to, capsules, tablets, granules, pills, pellets or microtablets.

Preferably, the solid dosage form is in a form of a microtablet.

More preferably, the microtablet may be further encapsulated in a capsule to be in a form of a microtablet-filled capsule.

According to the present invention, the “microtablet” means a single small tablet whose diameter is about less than or equal to 5 mm or whose weight is no more than 15 mg.

In the present invention, the manufacturing method of the pharmaceutical composition may further comprises a step (iii) film coating the solid dosage form of the pharmaceutical composition. The formulation of the coating film may be modified by adjusting the content of the film-coated material and the weight percentage ratio thereof relative to the pharmaceutical composition, depending on demands for different release rates. The film-coating material comprises one or more water-soluble or water non-soluble pharmaceutically acceptable polymer excipients, and the excipients may comprise, but are not limited to, ethyl cellulose, cellulose acetate, hydroxypropyl methylcellulose, methyl cellulose and/or hydroxypropyl methylcellulose phthalate. In the preferred embodiment of the present invention, the polymer excipient includes hydroxypropyl methylcellulose and/or ethyl cellulose.

The present invention provides a pharmaceutical composition, having a very wide range of the release rates, where the release rate ranges from, such as, very rapidly dissolving, rapidly dissolving, delayed release or controlled release, and either can be achieved. In other words, the drug release rate can be adjusted according to different conditions or requirements. In accordance with the dissolution test method(s) recited in “United States Pharmacopeia”, the drug release rate ranges between rapidly release over 85 wt. % of the at least one viscous active pharmaceutical ingredient or a pharmaceutically acceptable salt thereof in 15 minutes (classified as very rapidly dissolving) and controlled release with no less than 85 wt. % of the at least one viscous active pharmaceutical ingredient or a pharmaceutically acceptable salt thereof released after 24 hours (classified as controlled release).

The present invention further provides aforesaid manufacturing method of the pharmaceutical composition, which comprises steps of: (i) adding a starting material of the at least one viscous active pharmaceutical ingredient to a granulator, and performing granulation to the starting material to obtain granules of the at least one viscous active pharmaceutical ingredient; and (ii) making the granules of the at least one viscous active pharmaceutical ingredient obtained from the step (i) into a solid dosage form of the pharmaceutical composition individually or mixing the granules of the at least one viscous active pharmaceutical ingredient obtained from the step (i) with at least one pharmaceutically acceptable excipient into the solid dosage form of the pharmaceutical composition.

In the present invention, the manufacturing method may further comprise a step (iii) film coating the solid dosage form of the pharmaceutical composition.

The drug composition(s) of super high dosage load of the present invention may significantly reduce the content of excipients contained in the solid dosage forms, and the size of the solid dosage forms may also be decreased. More importantly, the said drug composition of the present invention can control the release rate depending on different demands and the optimal absorption rate and speed of the human body can be achieved.

Very little excipient(s) is needed for producing the solid dosage form of the pharmaceutical composition of the present invention, so that the content of the active pharmaceutical ingredient can be significantly increased, especially suitable for the pharmaceutical composition administered to patients with a high drug dosage.

Additionally, under the condition of the same content of active pharmaceutical ingredient, the size of the solid dosage form can be decreased for easy swallowing and to improve patient obedience in taking medicaments.

Furthermore, by adjusting the size of the solid dosage form (such as the size of the granules or the microtablet) or adding a small amount of excipient(s), the control of the drug rapid release rate of the medicine can be achieved. For example, increasing the specific surface areas of the microtablets to enhance the dissolution rate. Also, in general, the controlled release dosage form should adjust the formulation of tablets to achieve the effect of controlling the release rate. For example, selecting the release control capable excipient (such as the binder) to be mixed and granulated with the active pharmaceutical ingredients in the tablet production process. However, the present invention may achieve the effect of controlling release rate simply by adjusting the formulation or the weight ratio(s) of film coating. Thus, the aforesaid solid dosage form may further be coated with a polymer film coating to control the release rate of drugs.

### BRIEF DESCRIPTION OF THE DRAWINGS

The accompanying drawings are included to provide further understanding, and are incorporated in and constitute a part of this specification. The drawings illustrate exemplary embodiments and, together with the description, serve to explain the principles of the disclosure.

FIG. 1 shows the release profiles of the pharmaceutical compositions of Otilonium bromide with different HPMC content in the coating film, as described in Embodiment 5.

FIG. 2 is a graph illustrating the release profiles of the pharmaceutical compositions of Otilonium bromide with different coating weight gain, as described in Embodiment 6.

FIG. 3 is a graph illustrating the release profiles of the pharmaceutical compositions of Alfuzosin HCl/L-arginine α-ketoglutarate with different coating weight gain, as described in Embodiment 8.

### DESCRIPTION OF THE EMBODIMENTS

The present invention provides a high drug load pharmaceutical composition in order to increasing the flexibility of administering drugs to patients by medical personnel and overcoming patients' inconvenience of daily high-dose administration. The said pharmaceutical composition comprises active pharmaceutical ingredients, which have the physico-chemical properties of possessing a certain range of viscosity value. (The viscosity of the saturated aqueous solution of the active pharmaceutical ingredients must reach 20 cPs at 25° C. or above or the viscosity of the aqueous solution of the active pharmaceutical ingredients less than 55 wt. % is at least 10 cPs at 25° C.)

The present invention provides a high drug load pharmaceutical composition, which may further control dosage and release rate. The pharmaceutical composition may directly be made by using solvent to perform self granulation without adding a binder because that the active pharmaceutical ingredient has the certain range of viscosity value. Thus the drug load of the pharmaceutical composition is decrease and the species of excipients used is reduced substantially. The microtablets filled in the capsule may also be dispensed separately when there is a clinical need, and offer medical personnel and patients a drug choice with the more proper time and dosage to control disease perfectly.

Other features and advantages of the present invention will show in the beneficial embodiments and be claimed in the following paragraph.

### Embodiment

The following embodiments are used to demonstrate the present invention. The embodiments do not intend to limit the scope of the present invention in any way but indicate how to apply the materials and methods of the present invention.

So-called compounds which have a certain range of viscosity value in the embodiments of the present invention comprise Imatinib mesylate, Alfuzosin HCl, Otilonium bromide, L-arginine α-ketoglutarate, etc. So-called compounds which do not have a certain range of viscosity value (non-viscous) in the embodiments of the present invention comprise Metformin HCl, Hydrochlorothiazide, Levodopa, Sulpiride, etc.

The viscosities of saturated aqueous solutions or aqueous solutions with different weight percentage of the compounds are measured with Rotational viscometers (Brooksfield, USA) at 25° C. The aforementioned compounds are used as the active pharmaceutical ingredient (API) and granulation and tableting are performed to obtain the solid dosage form by using the manufacturing method of the present invention, and found that the compounds which have a certain range of viscosity value may proceed self granulation with solvent and be made into solid dosage form with high drug load (such as a tablet, which has the hardness between 3 kp and 8 kp, and the total brittleness is less than 0.1%); relatively, the compounds which do not have a certain range of viscosity value mostly could not proceed self granulation with solvent nor be made into solid dosage form with high drug load.

### Embodiment 1

### Comparison Between the Tablet-Forming Ability of APIs with Different Viscosity

Each API listed in Table 1-1 is separately weighed 1195 g and wet granulated in a high shear mixer using purified water. The wet granules are dried in an oven and sieved with 20 mesh screen. Each of the dried granules is separately transferred into a V-blender, and mixed with 5 g of magnesium stearate. The obtained mixture is compressed into tablets. Every tablet weighs about 100 mg, and contains magnesium stearate and an individual API.

In the embodiment, the APIs (Imatinib mesylate, Alfuzosin HCl, Otilonium bromide and L-arginine α-ketoglutarate) with high viscosity could self-granulate and be prepared as high drug-load tablets without the aid of binding agents. In contrast, some APIs showing low viscosity, such as Metformin HCl, Hydrochlorothiazide, Levodopa and Sulpiride can not be made into high milling resistance granules by self-granulation using water. Nor can they be made into tablets in the absence of excipients, such as binder or filler.—

TABLE 1-1
Viscosity and tablet properties of APIs. Testing tablets
comprise 99% of API and 1% magnesium stearate by weight.

| | Saturated | | | The tablet-forming | |
|---|---|---|---|---|---|
| | aqueous | Unsaturated aqueous | | ability | |
| Active | solution | solution at 25° C. | | Hard- | Friabil- |
| pharma- | at 25° C. | Weight | Viscos- | ness of | ity of |
| ceutical | Viscosity | percent | ity | tablets | tablets |
| ingredients | (cP) | (%) | (cP) | (kp) | (%) |
| Imatinib | about | 25 | about | 3-8 | <0.1 |
| mesylate | 1100 | | 15 | | |
| Alfuzosin | about | 30 | about | 3-8 | <0.1 |
| HCl | 170 | | 10 | | |
| Otilonium | about | 10 | about | 3-8 | <0.1 |
| bromide | 540 | | 12 | | |
| L-arginine | about | 55 | about | 3-8 | <0.1 |
| α-keto- | 30 | | 11 | | |
| glutarate | | | | | |
| Metformin | about | N/A | N/A | Failure of | N/A |
| HCl | 2 | | | forming | |
| | | | | intact | |
| | | | | tablets | |
| Hydrochloro- | about | N/A | N/A | Failure of | N/A |
| thiazide | 1 | | | self- | |
| | | | | granulation | |
| | | | | by water | |
| | | | | Failure of | |
| Levodopa | about | N/A | N/A | self- | N/A |
| | 1 | | | granulation | |
| | | | | by water | |
| | | | | Failure of | |
| Sulpiride | about | N/A | N/A | self- | N/A |
| | 2 | | | granulation | |
| | | | | by water | |

### Embodiment 2

### The Manufacturing and Dissolution Test of Different Imatinib Solid Dosage Forms

In the embodiment, several pharmaceutical compositions of Imatinib and the manufacturing process are provided as follows:

Each unit of formulation comprises Imatinib equivalent to 400 mg.

<Manufacturing method 1>: 1,195 g of the Imatinib mesylate powder is added into a V-blender and mixed with 5 g of magnesium stearate.

<Manufacturing method 2>: 1,195 g of Imatinib mesylate is added into a fluidized bed granulator and granulated with the purified water spray. The obtained granules are sieved and added into a V-blender, and then are evenly mixed with 5 g of magnesium stearate to form a mixture.

Formulation 2-1: The powder produced according to <Manufacturing method 1> (the powder weight of unit dose is 480 mg.)

Formulation 2-2: The powder produced according to <Manufacturing method 1> is filled into a hard gelatin capsule size 00 (the powder weight of unit dose is about 480 mg.)

Formulation 2-3: The mixture produced according to <Manufacturing method 2> is compressed into microtablets (Each microtablet weighs about 5.0 mg and 1.8 mm in size and the total weight of microtablets of unit dose is 480 mg.)

Formulation 2-4: The mixture produced according to <Manufacturing method 2> is compressed into microtablets (each microtablet weighs about 5.0 mg and 1.8 mm in size), and then filled into a hard gelatin capsule size 00 (the total weight of microtablets of unit dose is 480 mg.)

Formulation 2-5: The mixture produced according to <Manufacturing method 2> is compressed into tablets (the tablet weight of unit dose is 480 mg.)

Formulation 2-6: Commercial 400 mg Imatinib tablets (Glivec®. The tablet weight of unit dose is about 775 mg.)

The dissolution tests are conducted in a USP paddle apparatus 2 at 50 rpm, 37.0±0.5° C., using 900 mL of 0.1 N hydrochloric acid as the dissolution medium. The result is shown in Table 2-1.

TABLE 2-1
The dissolution test result of the pharmaceutical compositions
containing Imatinib equivalent to 400 mg unit dose.

| | Dissolution rate in 15 min (%) |
|---|---|
| Sample | Imatinib |
| Formulation 2-1 | 98.6 |
| Formulation 2-2 | 78.0 |
| Formulation 2-3 | 93.6 |
| Formulation 2-4 | 91.2 |
| Formulation 2-5 | 74.1 |
| Formulation 2-6 | 92.8 |

In table 2-1, Formulation 2-1, 2-3, 2-4 and 2-6 achieve very rapidly release in vitro, wherein the weight of Glivec® tablets is about 775 mg, the content of Imatinib is about 52% and contain excipients as follows: microcrystalline cellulose, hydroxypropyl methylcellulose, crospovidone, colloidal silicon dioxide, magnesium stearate, iron oxide red E172 and iron oxide yellow E172. The content of Imatinib in Formulation 2-3 (microtablets) and in the microtablet-filled capsules of Formulation 2-4 (microtablet-filled capsules) is up to 83%. By reducing the volume to increase the total surface area, the formulation of present invention may reach similar dissolution rate to the commercial product even with very little excipients. In addition, the dissolution rate of the microtablets (Formulation 2-3) or the microtablet-filled capsule (Formulation 2-4) is faster than that of the powder capsule (Formulation 2-2). It shows that the effect of the capsule to the dissolution rate must be considered when the solid dosage forms are designed. In the present embodiment, as compared with the powder, the microtablets may reduce contact area and interactions between the active pharmaceutical ingredient and the capsule. The higher density of microtablets would further benefit the development of high dosage level pharmaceutical compositions.

### Embodiment 3

### The Comparison of Dissolution Tests Between Different Imatinib Solid Dosage Forms

In the embodiment, the method of preparing three Imatinib solid dosage forms in different strengths (100 mg, 400 mg and 800 mg) is provided, which comprises the following steps:

A portion of 4780 g of Imatinib mesylate (equivalent to 4000 g of Imatinib) is dissolved in water to prepare an aqueous solution about 1% (w/v). The remaining Imatinib mesylate is added into a fluidized bed granulator and evenly granulated with the spray of said 1% aqueous solution.

The obtained granules are sieved and added into a V-blender, and then are evenly mixed with the excipients listed below to form a mixture. The mixture is compressed into tablets containing 100 mg, 400 mg and 800 mg of Imatinib (The weight of each tablet of said strength is 124 mg, 496 mg and 992 mg) or microtablets (each microtablet weighs about 4.0 mg and 1.8 mm in size). The microtablets may be administered directly or filled into capsules to adjust the dosage for clinical use. 100 mg of Imatinib equals to 31 microtablet, 400 mg of Imatinib equals to 124 microtablets, and 800 mg of Imatinib equals to 248 microtablets.

(a) Granular powder of Imatinib mesylate 4780 g

(b) Croscarmellose sodium 160 g

(c) Magnesium stearate 20 g

The dissolution tests are conducted in a USP paddle apparatus 2 at 50 rpm, 37.0±0.5° C., using 900 mL of 0.1 N hydrochloric acid as the dissolution medium. The test results are shown in Table 3-1, 3-2 and 3-3.

TABLE 3-1
The dissolution test results of the pharmaceutical compositions
containing Imatinib equivalent to 100 mg unit dose.

| | Dissolution rate in 15 min (%) |
|---|---|
| Sample | Imatinib |
| Microtablet-filled capsule | 95.1 |
| (Formulation 3-1) | |
| Tablet (Formulation 3-2) | 94.3 |

TABLE 3-2
The dissolution test results of the pharmaceutical compositions
containing Imatinib equivalent to 400 mg unit dose.

| | Dissolution rate in 15 min (%) |
|---|---|
| Sample | Imatinib |
| Microtablet-filled capsule | 92.4 |
| (Formulation 3-3) | |
| Tablet (Formulation 3-4) | 74.4 |

TABLE 3-3
The dissolution test results of the pharmaceutical compositions
containing Imatinib equivalent to 800 mg unit dose.

| | Dissolution rate in 15 min (%) |
|---|---|
| Sample | Imatinib |
| Microtablets (Formulation 3-5) | 94.6 |
| Tablet (Formulation 3-6) | 62.6 |

The results of Table 3-1, 3-2 and 3-3 show that, by using microtablets or microtablet-filled capsules, superior dissolving rate can be achieved while very little disintegrating agents or other excipients are added in the formulation. The Imatinib microtablets or microtablet-filled capsules exhibited rapid dissolving rate, while regular-sized tablets with the same formulation dissolved relatively slower, especially in high strengths (400 mg and 800 mg). Obviously, it is more necessary to administer high-dose drugs in the form of microtablet or microtablet-filled capsules so as to reach a better dissolution rate.

### Embodiment 4

### Manufacturing of 10,000 Units of Solid Dosage Form of Otilonium Bromide

In the embodiment, the methods of preparing Otilonium bromide solid dosage forms are provided, which comprise following steps:

1,200 g of Otilonium bromide is added into a fluidized bed granulator and granulated with the purified water spray. The obtained granules are sieved and added into a V-blender, and then are evenly mixed with 7 g of magnesium stearate to form a mixture. The mixture is compressed into tablets (each weighs about 120.7 mg) or microtablets (each weighs about 7.1 mg and 2.0 mm in size).

(a) Granular powder of Otilonium bromide 1,200 g

(b) Magnesium stearate 7 g

The dissolution tests are conducted in a USP paddle apparatus 2 at 50 rpm, 37.0±5° C., using 900 mL of 0.1 N hydrochloric acid as the dissolution medium. Each tested unit comprises Otilonium bromide equivalent to 120 mg. The results are shown in Table 4-1.

TABLE 4-1
The dissolution test result of the pharmaceutical compositions
containing 120 mg of Otilonium bromide per unit dose.

| | Dissolution rate in 15 min (%) |
|---|---|
| Sample | Otilonium bromide |
| Microtablet (Formulation 4-1) | 73.1 |
| Tablet (Formulation 4-2) | 59.7 |

### Embodiment 5

### Manufacturing of 10,000 Units of Solid Dosage Forms of Otilonium Bromide with Different Coating Formulation

In the embodiment, the methods of preparing Otilonium bromide solid dosage forms with different film coating solution are provided as follows:

1,200 g of Otilonium bromide is added into a fluidized bed granulator and granulated with the purified water spray. The obtained granules are sieved and added into a V-blender, and then are evenly mixed with 7 g of magnesium stearate to form a mixture. The mixture is compressed into microtablets (each weighs about 7.1 mg and 2.0 mm in size).

(a) Granular powder of Otilonium bromide 1,200 g

(b) Magnesium stearate 7 g

The microtablets obtained are added into a coater for film coating. Different compositions of coating solutions are separately listed in Table 5-1, wherein a 5.0% film weight gain in reached for the said microtablet core.

TABLE 5-1
Compositions of film coating solutions.

| Formulation | 5-1 | | 5-2 | | 5-3 | | 5-4 | | 5-5 | |
|---|---|---|---|---|---|---|---|---|---|---|
| Hydroxy- | — | | 0.2 | g | 0.4 | g | 0.6 | g | 0.8 | g |
| propyl | | | | | | | | | | |
| methyl- | | | | | | | | | | |
| cellulose | | | | | | | | | | |
| Ethyl | 80 | g | 79.8 | g | 79.6 | g | 79.4 | g | 79.2 | g |
| Cellulose | | | | | | | | | | |
| Dibutyl | 8 | g | 8 | g | 8 | g | 8 | g | 8 | g |
| sebacate | | | | | | | | | | |
| Pigment | q.s. | | q.s. | | q.s. | | q.s. | | q.s. | |
| 95% Ethanol | 4000 | g | 4000 | g | 4000 | g | 4000 | g | 4000 | g |

| | | | | | | | | | |
|---|---|---|---|---|---|---|---|---|---|
| Water | — | 150 | g | 300 | g | 450 | g | 600 | g |

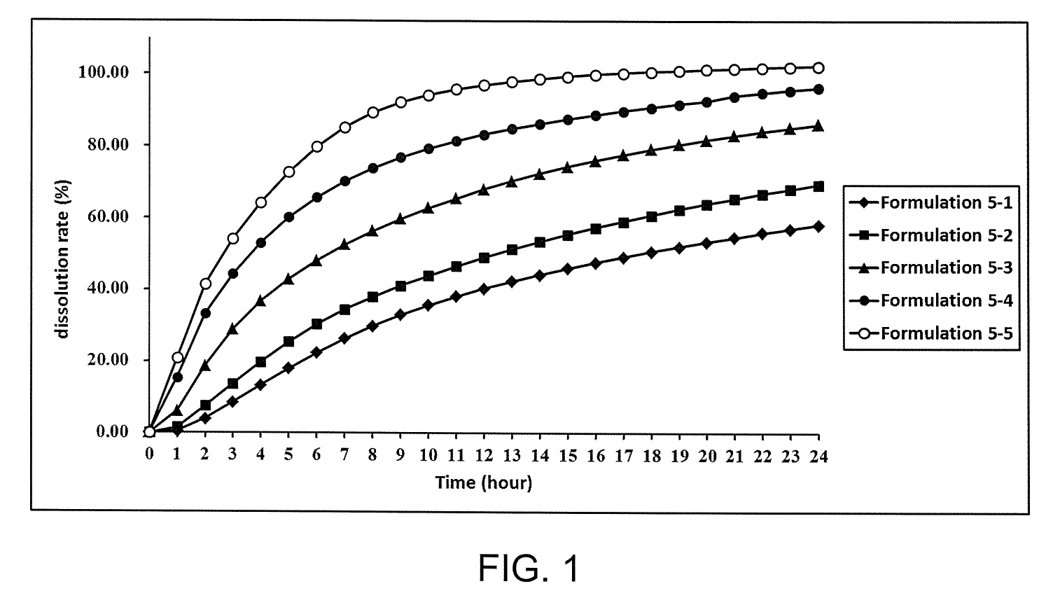

The dissolution tests are conducted in a USP paddle apparatus 2 at 50 rpm, 37.0±5° C., using 900 mL of 0.1 N hydrochloric acid as the dissolution medium. Each tested unit comprises Otilonium bromide equivalent to 120 mg. The results are shown in FIG. 1.

The content of porogen (Hydroxypropyl methylcellulose) is more, the dissolution rates is faster; when the content of porogen (Hydroxypropyl methylcellulose) is less, the release of active pharmaceutical ingredients are delayed. Thus, it can control drug release and present a very wide range of release rate only by regulating the formulations of film coating (for example, different content of porogen).

### Embodiment 6

### Manufacturing of 10,000 Units of Solid Dosage Form of Otilonium Bromide with Different Film Weight Gain

In the embodiment, the methods of preparing Otilonium bromide solid dosage forms with different coating film weight gain are provided as follows:

1,200 g of Otilonium bromide is added into a fluidized bed granulator\and granulated with the purified water spray. The obtained granules are sieved and added into a V-blender, and then are evenly mixed with 7 g of magnesium stearate to form a mixture. The mixture is compressed into microtablets (each weighs about 7.1 mg and 2.0 mm in size).

(a) Granular powder of Otilonium bromide 1,200 g

(b) Magnesium stearate 7 g

The microtablets obtained are added into a coater for film coating. The composition of film coating solution is listed in Table 6-1. Otilonium bromide microtablets are coated to a 2.5%-10% film weight gain.

Manufacture uncoated or coated microtablets of Formulation 6-1 to 6-5 according to the method above. Formulation 6-1 is a microtablet without film coating (the Formulation is equivalent to Formulation 4-1); Formulation 6-2 is a coated microtablet, wherein the weight of film coating is 2.5% of the said microtablet core; Formulation 6-3 is a coated microtablet, wherein the weight of film coating is 5.0% of the said microtablet core (the formulation is equivalent to Formulation 5-1); Formulation 6-4 is a coated microtablet, wherein the weight of film coating is 7.5% of the said microtablet core; Formulation 6-5 is a coated microtablet, wherein the weight of film coating is 10.0% of the said microtablet core.

TABLE 6-1
The composition of film coating solution.

| Formulation | Usage amount | |
|---|---|---|
| Hydroxypropyl methylcellulose | 0.8 | g |
| Ethyl Cellulose | 79.2 | g |
| Dibutyl sebacate | 8 | g |
| Pigment | q.s. | |
| 95% Ethanol | 4000 | g |
| Water | 600 | g |

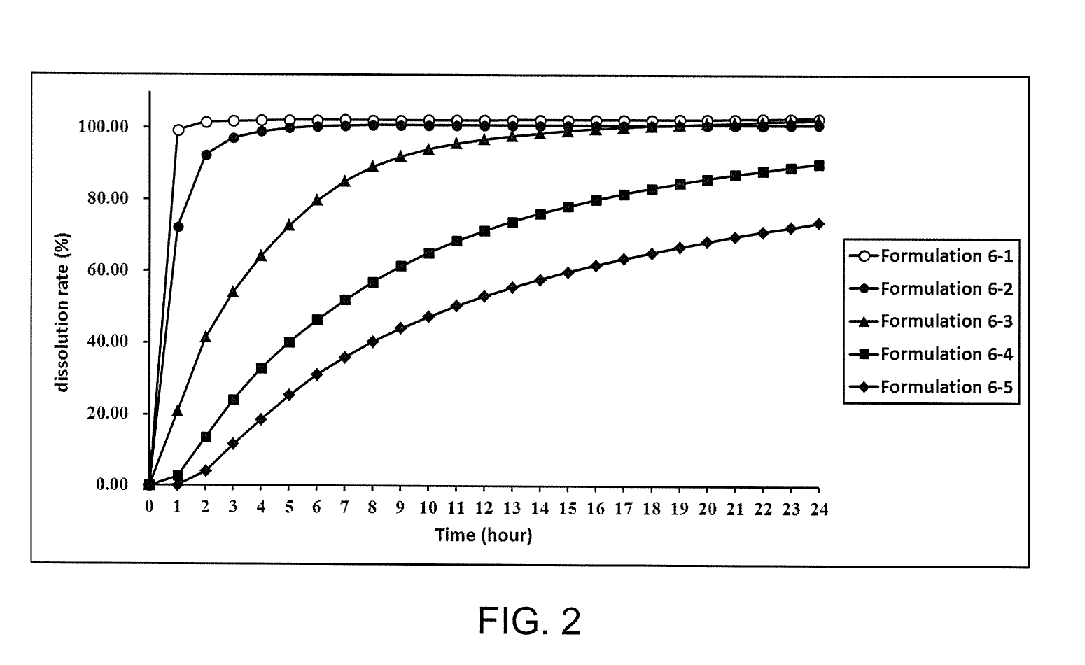

The dissolution tests are conducted in a USP paddle apparatus 2 at 50 rpm, 37.0±5° C., using 900 mL of 0.1 N hydrochloric acid as the dissolution medium. Each tested unit comprises Otilonium bromide equivalent to 120 mg. The test results are shown in FIG. 2.

As shown in FIG. 2, the weight of film coating is heavier, the release rate of active pharmaceutical ingredients is slower. For example, when the microtablet is not coated (Formulation 6-1), the active pharmaceutical ingredients thereof will be released over 90% in an hour; when the microtablet is coated with the film coating of 7.5% weight of the microtablet core (Formulation 6-4), the active pharmaceutical ingredients thereof will be released over 85% in 24 hours; when the microtablet is coated with the film coating of 10% weight of the microtablet core (Formulation 6-5), the active pharmaceutical ingredients thereof will not be released no more than 80% in 24 hours. Thus, in addition to regulating the content of porogen in the formulations of film coating, drug release may also be controlled through regulating the weight of film coating (namely accounting for different weight ratio of the microtablet core).

Generally speaking, in order to achieve the efficacy of controlled release, controlled-release tablets are not only prepared with film coating but also prepared by adding a great quantity of excipients to the core of tablet, such as binder and framework material. The present invention offer methods to select active pharmaceutical ingredients with specific viscosity, which may control release by only regulating formulations or weight ratio of film coating and without adding any binder or other controlled-release excipient. Additionally, the core of tablets are added with very small excipient in the present invention. As shown in Embodiment 5 and Embodiment 6, through the method of the present invention, the core of tablets may still present a very wide range of release rate under high dosage load condition.

### Embodiment 7

### Manufacturing of 10,000 Units of Solid Dosage Forms of Alfuzosin HCl and L-Arginine α-Ketoglutarate

In the embodiment, the methods of preparing Alfuzosin HCl/L-arginine α-ketoglutarate sold dosage forms are provided as follows:

All of 100 g of Alfuzosin HCl and 2,000 g of L-arginine α-ketoglutarate are added into a high shear mixer and granulated using purified water. The wet granules are dried in an oven and sieved with 20 mesh screen. The dried granules are transferred into a V-blender, and mixed with 30 g of magnesium stearate. The obtained mixture is compressed into tablets (each weighs about 213 mg) or microtablets (each weighs about 7.1 mg and 2.0 mm in size).

The dissolution tests are conducted in a USP paddle apparatus 2 at 50 rpm, 37.0±5° C., using 900 mL of 0.1 N hydrochloric acid as the dissolution medium. Each tested unit comprises Alfuzosin HCl equivalent to 10 mg and L-arginine α-ketoglutarate equivalent to 200 mg. The results are shown in Table 7-1.

TABLE 7-1
The dissolution test result of the pharmaceutical compositions
containing Alfuzosin HCl equivalent to 10 mg and L-arginine
α-ketoglutarate equivalent to 200 mg unit dose.

| | Dissolution rate in 15 min (%) |
|---|---|
| Sample | Alfuzosin HCl |
| Microtablets (Formulation 7-1) | 96.7 |
| Tablet (Formulation 7-2) | 82.4 |

### Embodiment 8

### Manufacturing of 10,000 Units of Solid Dosage Forms of Alfuzosin HCl/L-Arginine α-Ketoglutarate with Different Film Weight Gain

In the embodiment, the methods of preparing Alfuzosin HCl/L-arginine α-ketoglutarate solid dosage forms with different coating film weight gain are provided as follows:

All of 100 g of Alfuzosin HCl and 2,000 g of L-arginine α-ketoglutarate are added into a high shear mixer and granulated using purified water. The wet granules are dried in an oven and sieved with 20 mesh screen. The dried granules are transferred into a V-blender, and mixed with 30 g of magnesium stearate. The obtained mixture is compressed into microtablets (each weighs about 7.1 mg and 2.0 mm in size).

The microtablets obtained are placed into a coater (made by Yenchen Machinery Co., Ltd.) for film coating, and then controllable release coated microtablets are obtained. Formulations of film coating liquid are listed in Table 8-1. The weight of film coating in Formulation 8-1 is 5.0% of the said microtablet core; the weight of film coating in Formulation 8-2 is 10% of the said microtablet core; the weight of film coating in Formulation 8-3 is 15% of the said microtablet core; the weight of film coating in Formulation 8-4 is 20% of the said microtablet core.

TABLE 8-1
The composition of film coating solution.

| Formulation | Usage amount | |
|---|---|---|
| Hydroxypropyl methylcellulose | 0.2 | g |
| Ethyl Cellulose | 79.8 | g |
| Dibutyl sebacate | 8 | g |
| Pigment | q.s. | |
| 95% Ethanol | 4000 | g |
| Water | 150 | g |

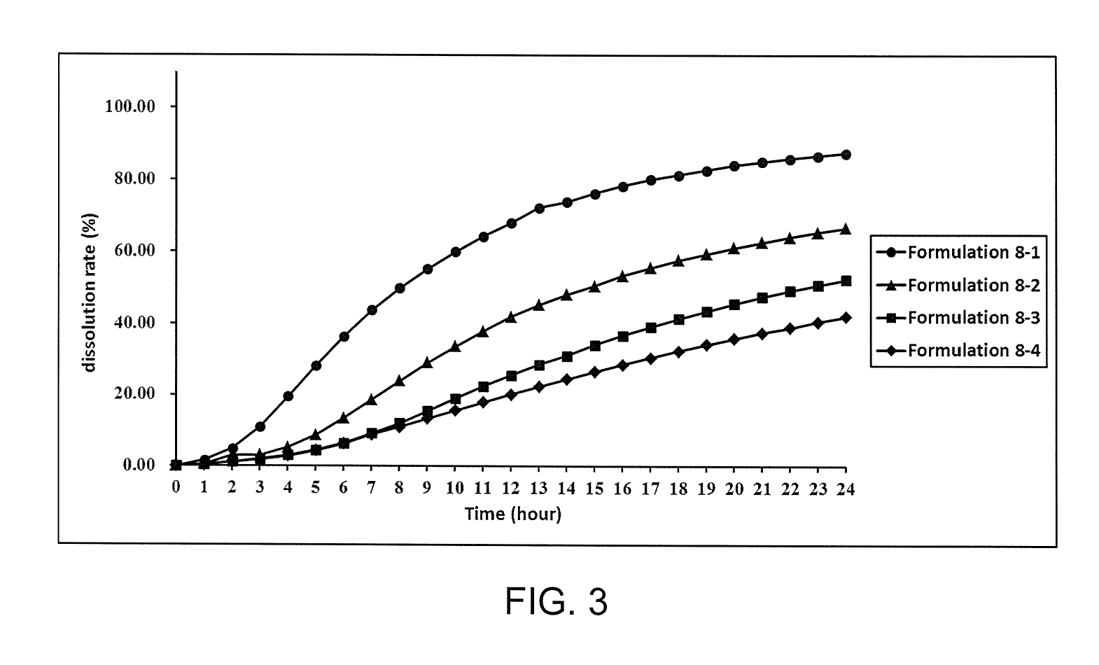

The dissolution tests are conducted in a USP paddle apparatus 2 at 50 rpm, 37.0±5° C., using 900 mL of 0.1 N hydrochloric acid as the dissolution medium. Each tested unit comprises Alfuzosin HCl equivalent to 10 mg and L-arginine α-ketoglutarate equivalent to 200 mg. The results are shown in FIG. 3.

According to Embodiment 7 and Embodiment 8, in the complex formulation, even though two active pharmaceutical ingredients have different viscosity characteristics, as long as at least one of them has outstanding viscosity, the viscous active pharmaceutical ingredient can be a binder and granulate with only water. Additionally, it still has good tableting efficiency while adding very little of excipient. As shown in FIG. 3, the release rate of active pharmaceutical ingredients is no more than 70% in 24 hours when the weight of film coating is over 10% of the said microtablet core (Formulation 8-2, 8-3 and 8-4). The formulation of film coating of Formulation 8-1 and Formulation 5-2 are the same and the weight of film coating of them are both 5.0% of the microtablet core. However, the release rate of Formulation 8-1 is significantly faster, which means that the same film coating of active pharmaceutical ingredient with different viscosity has different release profile.

The most innovative advantage of the present invention is to use the physicochemical property of active pharmaceutical ingredients of drugs or the salt (active pharmaceutical ingredient) thereof having a certain range of viscosity value, so that the active pharmaceutical ingredient can be a binder directly and the excipient may be reduced or free from adding. It is beneficial to produce solid granular by relatively simple process and further be formed into solid dosage form (such as granules and microtablets), which has super high drug load, to provide patients with the convenience and the possibility of administering a large amount of drugs. If the present invention are made into capsules, the stability of drugs will be increased because of air isolation by the capsule and the impurities will be reduced. For example, the disadvantage resulted from the hygroscopicity of the a crystalline form of Imatinib will be avoided. Using microtablet-filled capsules while administering mucous membrane stimulating drug may prevent the possible stimulation of esophageal mucosa. (It is common in pinaverium bromide drugs.) Besides, the microtablet-filled capsules are not only having characteristics of the capsules mentioned above but also make it more convenient for dosage adjustment by the way of the present invention. It is an important improvement for administering a variety of drugs which need to make dosage adjustment because of the physical demand of Asians.

The solid dosage form of the pharmaceutical compositions of the present invention has better stability than the common compositions in traditional pharmaceutics. And the pharmaceutical compositions mentioned in the present invention have simple formulations and have less compatibility considerations, so that the manufacturing process is easier and the cost is lower.

It will be apparent to those skilled in the art that various modifications and variations can be made to the structure of the present invention without departing from the scope or spirit of the invention. In view of the foregoing descriptions, it is intended that the present invention covers modifications and variations of this invention if they fall within the scope of the following claims and their equivalents.

What is claimed is:

1. A pharmaceutical composition, comprising at least one viscous active pharmaceutical ingredient or a pharmaceutically acceptable salt thereof, a content of the at least one viscous active pharmaceutical ingredient is at least 60 wt. %, relative to a total solid weight of the pharmaceutical composition.

2. The pharmaceutical composition according to claim 1, wherein a viscosity of a saturated aqueous solution of the at least one viscous active pharmaceutical ingredient is at least 20 cPs at 25° C.

3. The pharmaceutical composition according to claim 1, wherein a viscosity of an aqueous solution of less than 55 wt. % of the at least one viscous active pharmaceutical ingredient is at least 10 cPs at 25° C.

4. The pharmaceutical composition according to claim 1, wherein the at least one viscous active pharmaceutical ingredient is selected from the group consisting of tyrosine kinase inhibitors, selective al adrenoceptor antagonists, antimuscarinics, amino acid derivatives and the pharmaceutically acceptable salt thereof.

5. The pharmaceutical composition according to claim 4, wherein the tyrosine kinase inhibitor is Imatinib or a pharmaceutically acceptable salt thereof, the selective al adrenoceptor antagonist is Alfuzosin or a pharmaceutically acceptable salt thereof, the antimuscarinics is Otilonium or a pharmaceutically acceptable salt thereof, and the amino acid derivative is L-arginine α-ketoglutarate.

6. The pharmaceutical composition according to claim 1, wherein a drug release rate of the pharmaceutical composition ranges between a rapidly release over 85 wt. % of the at least one viscous active pharmaceutical ingredient in 15 minutes and controlled release with no less than 85 wt. % of the at least one viscous active pharmaceutical ingredient after 24 hours.

7. A method for manufacturing a pharmaceutical composition of claim 1, comprising:
(i) adding a starting material of the at least one viscous active pharmaceutical ingredient to a granulator, and performing granulation to the starting material to obtain granules of the at least one viscous active pharmaceutical ingredient; and
(ii) making the granules of the at least one viscous active pharmaceutical ingredient obtained from the step (i) into a solid dosage form of the pharmaceutical composition individually or mixing the granules of the at least one viscous active pharmaceutical ingredient obtained from the step (i) with at least one pharmaceutically acceptable excipient into the solid dosage form of the pharmaceutical composition.

8. The method according to claim 7, wherein the method after the step (ii) further comprises a step (iii) film coating the solid dosage form of the pharmaceutical composition.

9. A pharmaceutical composition, comprising at least one viscous active pharmaceutical ingredient or a pharmaceutically acceptable salt thereof, a content of the at least one viscous active pharmaceutical ingredient or a pharmaceutically acceptable salt thereof is at least 60 wt. %, relative to a total solid weight of the pharmaceutical composition, wherein the pharmaceutical composition is prepared by a manufacturing method, comprising steps of:
(i) adding a starting material of the at least one viscous active pharmaceutical ingredient to a granulator, and performing granulation to the starting material to obtain the granules of the at least one viscous active pharmaceutical ingredient; and
(ii) making the granules of the at least one viscous active pharmaceutical ingredient obtained from the step (i) into a solid dosage form of the pharmaceutical composition individually or mixing the granules of the at least one viscous active pharmaceutical ingredient obtained from the step (i) with at least one pharmaceutically acceptable excipient into the solid dosage form of the pharmaceutical composition.

10. The pharmaceutical composition according to claim 9, wherein a viscosity of a saturated aqueous solution of the at least one viscous active pharmaceutical ingredient is at least 20 cPs at 25° C.

11. The pharmaceutical composition according to claim 9, wherein a viscosity of an aqueous solution of less than 55 wt. % of the at least one viscous active pharmaceutical ingredient is at least 10 cPs at 25° C.

12. The pharmaceutical composition according to claim 9, wherein the manufacturing method further comprises a step (iii) film coating the solid dosage form of the pharmaceutical composition.

13. The pharmaceutical composition according to claim 12, wherein a film coating material used in the step (iii) comprises one or more water-soluble or water insoluble pharmaceutically acceptable polymer excipients.

14. The pharmaceutical composition according to claim 9, wherein the granulation of the starting material in the step (i) comprises applying wet granulation by using a high shear mixer, applying dry granulation by using a roller compactor or applying spray granulation by using a fluidized bed granulator.

15. The pharmaceutical composition according to claim 9, wherein the at least one pharmaceutically acceptable excipient is selected from the group consisting of disintegrants, glidants, lubricants and combinations thereof.

16. The pharmaceutical composition according to claim 9, wherein the content of the at least one pharmaceutically acceptable excipient ranges from 0.1 wt. % to 15 wt. %, relative to the total solid weight of the pharmaceutical composition.

17. The pharmaceutical composition according to claim 9, wherein the solid dosage form is in a form of a capsule, a tablet, granules, pills, pellets or microtablets.

18. The pharmaceutical composition according to claim 9, wherein the solid dosage form is in a form of microtablets.

19. The pharmaceutical composition according to claim 18, wherein the microtablets are encapsulated in a capsule to form a microtablet-filled capsule.

20. The pharmaceutical composition according to claim 18, wherein a diameter of any one of the microtablets is no more than 5 mm.

21. The pharmaceutical composition according to claim 18, wherein a weight of any one of the microtablets is no more than 15 mg.

22. The pharmaceutical composition according to claim 9, wherein the at least one viscous active pharmaceutical ingredient is used for treating human malignant or nonmalignant hyperplasia, benign prostatic hyperplasia, dysuresia, functional gastrointestinal disorders or liver diseases or as a nutritional supplement.

23. The pharmaceutical composition according to claim 9, wherein the at least one viscous active pharmaceutical ingredient is selected from the group consisting of tyrosine kinase inhibitors, selective al adrenoceptor antagonists, antimuscarinics, amino acid derivatives and the pharmaceutically acceptable salt thereof.

24. The pharmaceutical composition according to claim 23, wherein the tyrosine kinase inhibitor is Imatinib or a pharmaceutically acceptable salt thereof, the selective al adrenoceptor antagonist is Alfuzosin or a pharmaceutically acceptable salt thereof, the antimuscarinics is Otilonium or a pharmaceutically acceptable salt thereof, and the amino acid derivative is L-arginine α-ketoglutarate.

25. The pharmaceutical composition according to claim 9, wherein a drug release rate of the pharmaceutical composition ranges between a rapidly release over 85 wt. % of the at least one viscous active pharmaceutical ingredient in 15 minutes and controlled release with no less than 85 wt. % of the at least one viscous active pharmaceutical ingredient after 24 hours.

26. The pharmaceutical composition according to claim 12, wherein a drug release rate of the pharmaceutical composition ranges between a rapidly release over 85 wt. % of the at least one viscous active pharmaceutical ingredient in 15 minutes and controlled release with no less than 85 wt. % of the at least one viscous active pharmaceutical ingredient after 24 hours.

27. The pharmaceutical composition according to claim 18, wherein a drug release rate of the pharmaceutical composition ranges between a rapidly release over 85 wt. % of the at least one viscous active pharmaceutical ingredient in 15 minutes and controlled release with no less than 85 wt. % of the at least one viscous active pharmaceutical ingredient after 24 hours.
